(19) United States
(12) Patent Application Publication
(10) Pub. No.: US 2024/0000772 A1
(43) Pub. Date: Jan. 4, 2024

(54) MICROSPHERE FORMULATIONS COMPRISING NALTREXONE AND METHODS FOR MAKING AND USING THE SAME

(71) Applicant: Oakwood Laboratories, LLC, Oakwood Village, OH (US)

(72) Inventors: Bradley Minrovic, Willoughby, OH (US); Emma Murphy, Kent, OH (US); Kaitlin Zemanski, Munroe Falls, OH (US); Tracy Richey, Stow, OH (US); Michaela Giltner, Akron, OH (US)

(21) Appl. No.: 18/467,283

(22) Filed: Sep. 14, 2023

Related U.S. Application Data
(63) Continuation-in-part of application No. PCT/US22/70941, filed on Mar. 3, 2022.
(60) Provisional application No. 63/266,660, filed on Jan. 11, 2022.
(60) Provisional application No. 63/161,159, filed on Mar. 15, 2021.
(60) Provisional application No. 63/161,187, filed on Mar. 15, 2021.
(60) Provisional application No. 63/483,100, filed on Feb. 3, 2023.
(60) Provisional application No. 63/510,966, filed on Jun. 29, 2023.

(51) Int. Cl. A61K 31/485; A61K 9/16

(52) U.S. Cl. CPC A61K 31/485; A61K 9/1647

(57) ABSTRACT

Microsphere formulations comprising naltrexone are provided. Methods of making and using the microsphere formulations are also provided.

### CROSS-REFERENCE TO RELATED APPLICATIONS

This application is a continuation in part of International Application No. PCT/US22/70941, filed on Mar. 3, 2022, which claims the benefit of U.S. Provisional Patent Application No. 63/266,660, filed on Jan. 11, 2022, U.S. Provisional Patent Application No. 63/161,159, filed on Mar. 15, 2021, and U.S. Provisional Patent Application No. 63/161,187, filed on Mar. 15, 2021. This application also claims the benefit of U.S. Provisional Patent Application No. 63/483,100, filed on Feb. 3, 2023, and U.S. Provisional Patent Application No. 63/510,966, filed on Jun. 29, 2023. Each of these applications is incorporated by reference herein in its entirety.

### BACKGROUND

Naltrexone (chemical formula C₂₀H₂₃NO₄; CAS Number 16590-41-3), characterized by the general structure:

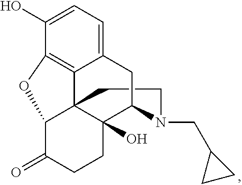 [SMILES: O=C1CC[C@@]2(O)[C@H]3Cc4ccc(O)c5c4[C@@]2(CCN3CC2CC2)[C@H]1O5]

is a medication used to treat alcohol and opioid dependence. Naltrexone is currently orally administered as a tablet or injected into a muscle. Naltrexone is commercially available under the trade name Vivitrol®.

Vivitrol® is a once-per-month extended-release microsphere formulation wherein naltrexone is encapsulated in a poly(D,L-lactide-co-glycolide), 75:25 polymer matrix, having a drug load of approximately 33.7% and a particle size of approximately 81 μm (D₅₀). Vivitrol® must not be administered intravenously or subcutaneously. Some patients experience side effects from using Vivitrol® and may require another treatment option. Thus, a need exists for an alternative extended-release naltrexone-encapsulating microsphere formulation, especially one having a high drug load (>40% by weight), small particle size (about 20-60 μm (D₅₀)), long release duration (≥˜30, 60, 90, or even 120 days), and a different mode of release.

### SUMMARY

Microsphere formulations comprising naltrexone are provided. The microsphere formulations comprise polymer microspheres, each polymer microsphere comprising: (i) naltrexone; and (ii) a biodegradable polymer comprising either a poly(ortho ester) polymer (a “POE”) or a poly(D,L-lactide) polymer (a “PLA”), wherein each polymer microsphere comprises a drug load of naltrexone of greater than 40% by weight of the polymer microsphere, and wherein the polymer microspheres have a particle size of about 20 μm to about 60 μm (D₅₀), with the proviso that the biodegradable polymer does not include a poly(D,L-lactide-co-glycolide) (a “PLGA”). In one aspect, the microsphere formulations are characterized in that the naltrexone is released over a period of about 60 days. In other aspects, the microsphere formulations are characterized in that the naltrexone is released over a period of about 90 days. In other aspects, the microsphere formulations are characterized in that the naltrexone is released over a period of about 120 days. In another aspect, the microsphere formulations are characterized in that they have a low initial burst release, that is, not more than 20% of the naltrexone is released within about 24 hours of injection into a subject.

In one aspect, the microsphere formulations may be made by a method, the method comprising: (A) mixing: (i) the biodegradable polymer comprising a POE or a PLA; (ii) a primary solvent; (iii) naltrexone; and (iv) a co-solvent, to form a dispersed phase; (B) mixing: (i) water; and (ii) a surfactant, to form a continuous phase; and (C) combining the dispersed phase with the continuous phase in a homogenizer.

In one aspect, a method for treating alcohol and/or opioid dependence is provided. The method may comprise administering by intramuscular or subcutaneous injection to a patient in need thereof a microsphere formulation made according to the methods described herein, wherein the formulation is administered to the patient with a dosing schedule of about every 60, 90, or 120 days.

In another aspect, use is disclosed of a microsphere formulation comprising polymer microspheres, each polymer microsphere comprising: (i) naltrexone; and (ii) a biodegradable polymer comprising a POE or a PLA, wherein each polymer microsphere comprises a drug load of naltrexone of greater than 40% by weight of the polymer microsphere, and wherein the polymer microspheres have a particle size of about 20 μm to about 60 μm (D₅₀), in the manufacture of a medicament for the treatment of alcohol and/or opioid dependence.

In another aspect, a microsphere formulation comprising polymer microspheres, each polymer microsphere comprising: (i) naltrexone; and (ii) a biodegradable polymer comprising a POE or a PLA, wherein each polymer microsphere comprises a drug load of naltrexone of greater than 40% by weight of the polymer microsphere, and wherein the polymer microspheres have a particle size of about 20 μm to about 60 μm (D₅₀), is provided for use as a medicament for the treatment of alcohol and/or opioid dependence.

In another aspect, a kit is provided, the kit comprising polymer microspheres, each polymer microsphere comprising: (i) naltrexone; and (ii) a biodegradable polymer comprising a POE or a PLA, wherein each polymer microsphere comprises a drug load of naltrexone of greater than 40% by weight of the polymer microsphere, and wherein the polymer microspheres have a particle size of about 20 μm to about 60 μm (D₅₀).

### BRIEF DESCRIPTION OF THE FIGURES

FIG. 1 is a schematic depicting a method for making naltrexone-encapsulated polymer microspheres.

FIG. 2 is a graph showing naltrexone release over time in dogs from naltrexone-encapsulating PLA-based polymer microspheres in direct comparison to Vivitrol®.

FIG. 3 is a graph showing naltrexone release over time in dogs from naltrexone-encapsulating POE-based polymer microspheres in direct comparison to Vivitrol®.

FIG. 4 is a graph showing in vivo release profiles of several naltrexone-encapsulating polymer microspheres.

FIG. 5 is an exploded view of the graph shown in FIG. 4 and demonstrates the relative “burst” profiles of the naltrexone-encapsulating polymer microspheres.

### DETAILED DESCRIPTION

Microsphere formulations comprising naltrexone are provided. The microsphere formulations comprise polymer microspheres, each polymer microsphere comprising: (i) naltrexone; and (ii) a biodegradable polymer comprising a POE or a PLA, wherein each polymer microsphere comprises a drug load of naltrexone of greater than 40% by weight of the polymer microsphere, and wherein the polymer microspheres have a particle size of about 20 μm to about 60 μm (D₅₀).

In one aspect, the microsphere formulations may be made by a method, the method comprising: (A) mixing: (i) the biodegradable polymer comprising a POE or a PLA, but not a PLGA; (ii) a primary solvent; (iii) naltrexone; and (iv) a co-solvent, to form a dispersed phase; (B) mixing: (i) water; and (ii) a surfactant, to form a continuous phase; and (C) combining the dispersed phase with the continuous phase in a homogenizer.

### Naltrexone

In one aspect, the naltrexone is a free base. In one aspect, the naltrexone has a dichloromethane (“DCM”) solubility of 100 mg/mL, ethyl acetate (“EA”) solubility of 26 mg/mL, and benzyl alcohol (“BA”) solubility of >250 mg/mL. In one aspect, the naltrexone has a pKa=8.4.

In another aspect, the naltrexone is an HCl salt. In one aspect, the naltrexone HCl salt has a water solubility of about 100 mg/mL.

### Biodegradable Polymers

In one aspect, the biodegradable polymer is a POE. POEs release through surface degradation, as compared to PLGAs, which release by bulk hydrolysis. Suitable POE polymers or co-polymers may include a cyclohexanedimethanol:triethylene glycol (CHDM:TEG) co-polymer, a cyclohexanedimethanol:triethylene glycol:triethylene glycol glycolide (CHDM:TEG:TEG-GL) tri-block polymer, a 3,9 -Diethylidene-2,4,8,10-tetraoxaspiro[5.5]undecane:triethylene glycol (DETOSU:TEG) polymer, or a 3,9-Diethylidene-2,4,8,10-tetraoxaspiro[5.5] undecane:triethylene glycol:triethylene glycol glycolide (DETOSU:TEG:TEG-GL) polymer. In one aspect, the CHDM:TEG ratio may be about 93:7, with a molecular weight of about 22 kDa. In another aspect, the CHDM:TEG ratio may be about 80:20, with a molecular weight of about 26 kDa. In one aspect, the CHDM:TEG:TEG-GL ratio may be about 88:10:2, with a molecular weight of about 27 kDa. In another aspect, the CHDM:TEG:TEG-GL ratio may be about 70:0:30, with a molecular weight of about 20 kDa.

In one aspect, the biodegradable polymer is a PLA. The PLA may have an inherent viscosity of about 0.15 dL/g to about 0.75 dL/g, including from about 0.15 dL/g to about 0.25 dL/g, from about 0.26 dL/g to about 0.54 dL/g, including 0.36 dL/g, and from about 0.55 dL/g to about 0.75 dL/g. In one aspect, the PLA comprises Lactel® DL-PLA, ester terminated, IV=0.36 dL/g, MW=46 kDa, supplied by Evonik Industries AG (“DL-PLA”). In one aspect, PLGA polymers are specifically excluded.

### Dispersed Phase

In one aspect, the dispersed phase comprises a primary solvent. In one aspect, the primary solvent comprises DCM. The dispersed phase may also include up to about 50% by weight of a co-solvent capable of optimizing the solubility of naltrexone in the dispersed phase. In one aspect, the co-solvent may be BA, dimethyl sulfoxide, dimethyl formamide, dimethyl acetamide, acetonitrile, ethanol, N-methyl pyrrolidone, EA, or any other solvent that increases the solubility of naltrexone in the dispersed phase. In one aspect, the primary solvent comprises DCM, and the co-solvent comprises BA. In one aspect, the ratio of DCM to BA is about 3:1. The organic solvent is removed from the microspheres in the course of their preparation. A microsphere is considered to be “essentially free” of organic solvent if the microsphere meets the standards set forth in the “ICH Harmonised Guideline, Impurities: Guideline for Residual Solvents Q3C(R8), Current Step 4 version dated 22 Apr. 2021,” which is incorporated herein by reference in its entirety.

### Continuous Phase

The dispersed phase may be combined with an aqueous continuous phase that comprises water and, optionally, a surfactant. In one aspect, the continuous phase has a pH of about 6.

The surfactant component may be present in the continuous phase in an amount of about 0.35% to about 1.0% by weight in water. In one aspect, the surfactant component comprises polyvinyl alcohol (“PVA”) in a concentration of about 0.35% by weight in water.

In some aspects, the dispersed phase flow rate to the homogenizer may be from about mL/min to about 30 mL/min, including about 20 mL/min and about 25 mL/min. In some aspects, the continuous phase flow rate to the homogenizer may be about 2 L/min. Thus, in one aspect, the continuous phase:dispersed phase ratio may be from about 66:1 to about 200:1, including about 100:1 and about 80:1.

The continuous phase may be provided at room temperature or above or below room temperature. In some aspects, the continuous phase may be provided at about 40° C., about 37° C., about 35° C., about 30° C., about 25° C., about 20° C., about 15° C., about 10° C., about 5° C., about 0° C., and any range or value between any of those temperature values.

### Homogenizer

For brevity, and because the methods are equally applicable to either, the phrase “homogenizer” contemplates a system or apparatus that can homogenize the dispersed phase and the continuous phase, emulsify the dispersed phase and the continuous phase, or both, which systems and apparatuses are known in the art. For example, in one aspect, the homogenizer is an in-line Silverson Homogenizer (commercially available from Silverson Machines, Waterside, UK) or a Levitronix® BPS-i100 integrated pump system used, e.g., as described in U.S. Pat. No. 11,167,256, which is incorporated by reference herein in its entirety. In one aspect, the homogenizer is a membrane emulsifier. In one aspect, the homogenizer runs at an impeller speed of about 1,000 to about 4,000 revolutions per minute (“RPM”), including about 1,250 RPM, about 1,750 RPM, about 2,000 RPM, about 3,250 RPM, or any value or range between any of those RPM values.

### Drug Load

The drug load of each polymer microsphere in a drug to polymer ratio, expressed as a percentage, may be greater than 40 wt/wt %, about 45 wt/wt %, about 50 wt/wt %, about 55 wt/wt %, about 60 wt/wt %, from 40 wt/wt % to 60 wt/wt %, from 50 wt/wt % to 60 wt/wt %, or any value or range between any of those percentages.

### Particle Size

The polymer microspheres may be any size that is safely and efficaciously injectable. In one aspect, the polymer microspheres may have a particle size between about 20 μm (D₅₀) and about 60 μm (D₅₀), between about 20 μm (D₅₀) and about 35 μm (D₅₀), between about 20 μm (D₅₀) and about 45 μm (D₅₀), between about 25 μm (D₅₀) and about 50 μm (D₅₀), between about 30 μm (D₅₀) and about 45 μm (D₅₀), between about 45 μm (D₅₀) and about 60 μm (D₅₀), less than about 55 μm (D₅₀), and less than 60 μm (D₅₀), or any value or range between any of those particle sizes.

### Extended Release

Where the polymer is a PLA, the microsphere formulations may be characterized in that they have a duration of release of at least about two weeks and up to about twelve weeks. In some aspects, the microsphere formulations have a duration of release of about three weeks, about four weeks, about five weeks, and about six weeks. In some aspects, the duration of release is about 30 days.

Where the polymer is a POE comprising CHDM:TEG with a ratio of about 93:7, or the polymer is a POE comprising CHDM:TEG:TEG-GL with a ratio of about 88:10:2, the microsphere formulations may be characterized in that they may have a duration of release of at least about 60 days, including up to about 100-120 days, or any value or range between any of those release durations. In one aspect, Batch No. 3 may be characterized in that it has a duration of release of about 120 days.

Where the polymer is a POE comprising CHDM:TEG:TEG-GL with a ratio of about the microsphere formulations may be characterized in that they have a duration of release of about 30 days, about 60 days, about 90 days, or about 120 days, or any value or range between any of those release durations.

Where the polymer is a POE comprising CHDM:TEG with a ratio of about 80:20, the microsphere formulations may be characterized in that they have a duration of release of about 60, about 90 days, or about 120 days, or any value or range between any of those release durations. In one aspect, Batch No. 11 may be characterized in that it has a duration of release of about 60 days. In one aspect, Batch No. 12 may be characterized in that it has a duration of release of about days. In one aspect, Batch No. 9 may be characterized in that it has a duration of release of about 120 days.

In some aspects, the microsphere formulations are further characterized in that they have a low initial burst release, that is, not more than 20% of the naltrexone is released within about 24 hours of injection into a subject.

### Therapeutic Benefits

In one aspect, a method for treating alcohol and/or opioid dependence is provided. The method may comprise administering by intramuscular or subcutaneous injection to a patient in need thereof a microsphere formulation made according to the methods described herein, wherein the formulation is administered to the patient with a dosing schedule of about every 60, 90, or 120 days.

In another aspect, use is disclosed of a microsphere formulation comprising polymer microspheres, each polymer microsphere comprising: (i) naltrexone; and (ii) a biodegradable polymer comprising a POE or a PLA, wherein each polymer microsphere comprises a drug load of naltrexone of greater than 40% by weight of the polymer microsphere, and wherein the polymer microspheres have a particle size of about 20 μm to about 60 μm (D₅₀), in the manufacture of a medicament for the treatment of alcohol and/or opioid dependence.

In another aspect, a microsphere formulation comprising polymer microspheres, each polymer microsphere comprising: (i) naltrexone; and (ii) a biodegradable polymer comprising a POE or a PLA, wherein each polymer microsphere comprises a drug load of naltrexone of greater than 40% by weight of the polymer microsphere, and wherein the polymer microspheres have a particle size of about 20 μm to about 60 μm (D₅₀), is provided for use as a medicament for the treatment of alcohol and/or opioid dependence.

In another aspect, a kit is provided, the kit comprising polymer microspheres, each polymer microsphere comprising: (i) naltrexone; and (ii) a biodegradable polymer comprising a POE or a PLA, wherein each polymer microsphere comprises a drug load of naltrexone of greater than 40% by weight of the polymer microsphere, and wherein the polymer microspheres have a particle size of about 20 μm to about 60 μm (D₅₀).

### EXAMPLES

### Example 1—General Preparation of Polymer Microspheres Comprising Naltrexone

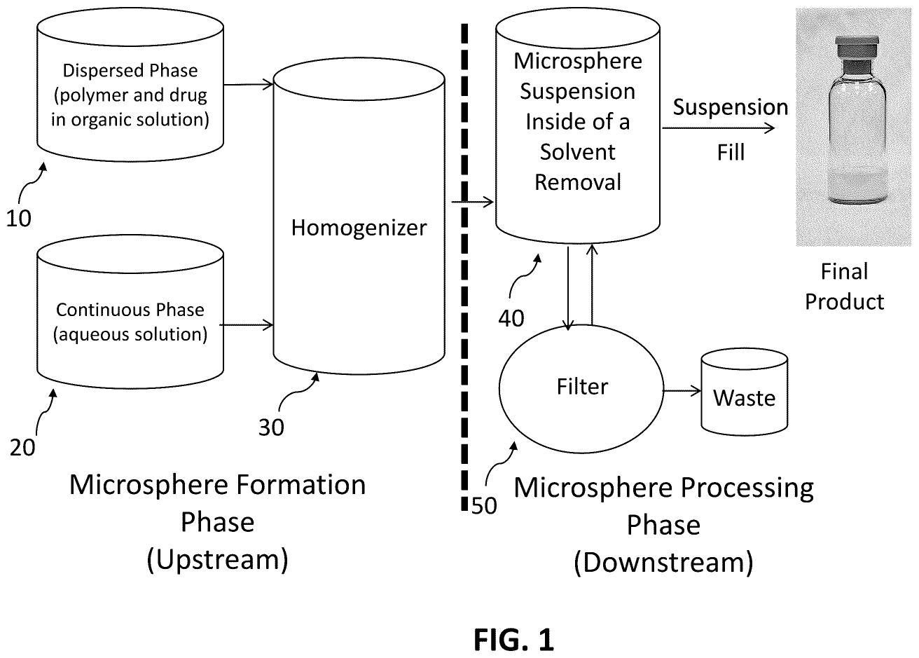

Microsphere Formation Phase. With reference to FIG. 1, a dispersed phase (“DP”) 10 is formed by dissolving a polymer matrix (such as a POE or PLA polymer) in an organic solvent system (such as DCM and BA), followed by the addition of naltrexone with mixing until completely dissolved. The DP 10 is filtered using a 0.2 μm sterilizing PTFE or PVDF membrane filter (such as EMFLON, commercially available from Pall or SartoriousAG) and pumped into a homogenizer 30 at a defined flow rate. A continuous phase (“CP”) 20 comprising water and surfactant is also pumped into the homogenizer 30 at a defined flow rate. The speed of the homogenizer 30 is generally fixed to achieve a desired polymer microsphere size distribution. A representative continuous “upstream” microsphere formation phase is described in U.S. Pat. No. which is incorporated by reference herein in its entirety.

Microsphere Processing Phase. The formed or forming microspheres exit the homogenizer 30 and enter a solvent removal vessel (“SRV”) 40. Water may be added to the SRV during microsphere formation to minimize the solvent level in the aqueous medium. See, e.g., U.S. Pat. No. 9,017,715, which is incorporated by reference herein in its entirety. After the DP has been exhausted, the CP and water flow rates are stopped, and the washing steps are initiated. Solvent removal is achieved using water washing and a hollow fiber filter (commercially available as HFF from Cytiva) 50. A representative “downstream” microsphere processing phase is described in U.S. Pat. No. 6,270,802, which is incorporated by reference herein in its entirety.

The washed microspheres are collected and freeze-dried overnight in a lyophilizer (Virtis) to remove any moisture. The resulting microspheres are a free-flowing off-white bulk powder.

### Example 2—Preparation of Naltrexone-Encapsulated PLA Polymer Microspheres—Batch 1

Following the general procedure described in Example 1, illustrated in FIG. 1, and detailed in Table 1, the DP was formed by dissolving 13.5 g of DL-PLA polymer in 59.4 g of DCM and 19.8 g of BA (DCM/BA (3:1)), followed by addition of naltrexone (16.5 g) with mixing until completely dissolved. The DP was filtered and pumped at a flow rate of 25 mL/min into a Levitronix® BPS-i100 integrated pump system operating at 3,250 RPM. The CP comprising PVA was also pumped into the homogenizer at a flow rate of 2 L/min (CP:DP=80:1).

The formed or forming microspheres exited the homogenizer and entered the SRV. Deionized water was added to the SRV. Solvent removal was achieved using water washing and a hollow fiber filter. The bulk suspension was collected via filtration and lyophilized to obtain a free-flowing powder.

The process parameters and the characterization data for a representative batch (Batch #1) are shown in Table 1 in comparison to Vivitrol®:

TABLE 1

| Batch # | 1 | Vivitrol ® |
|---|---|---|
| Polymer | DL-PLA | PLGA 75:25 |
| Polymer IV (dL/g) | 0.36 | Unknown |
| Solvent System | DCM/BA (3:1) | Unknown/BA |
| Homogenizer RPM | 3,250 | N/A |
| Drug Load (%) | 47.5 | 33.7 |
| Residual Solvents | 0.1/2.0 | N.D./0.6 |
| (% wt.) | | |
| Particle Size | 15 | 50 |
| (D₁₀) | | |
| Particle Size | 31 | 81 |
| (D₅₀) | | |
| Particle Size | 53 | 129 |
| (D₉₀) | | |
| Microsphere MW (kDa) | 41 | 74 |

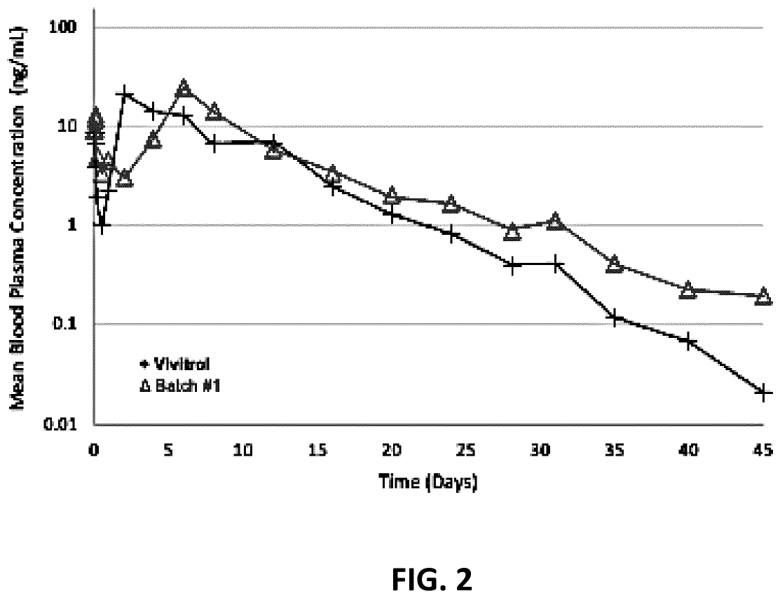

FIG. 2 is a graph showing naltrexone release over time in dogs from naltrexone-encapsulating PLA polymer microspheres in direct comparison to Vivitrol®.

### Example 3—Preparation of Naltrexone-Encapsulated POE Polymer Microspheres—Batches 2-4

Following the general procedure described in Example 1, illustrated in FIG. 1, and detailed in Table 2, the DP was formed by dissolving 13.5 g of POE in 59.4 g of DCM and 19.8 g of BA (DCM/BA (3:1)), followed by addition of naltrexone (16.5 g) with mixing until completely dissolved. The DP was filtered and pumped at a flow rate of 25 mL/min into a Levitronix® BPS-i100 integrated pump system operating at 3,250 RPM (Batches 2 and 3) or 4,000 RPM (Batch 4). The CP comprising 0.35% PVA was also pumped into the homogenizer at a flow rate of 2 L/min (CP:DP=80:1).

The formed or forming microspheres exited the homogenizer and entered the SRV. Deionized water was added to the SRV. Solvent removal was achieved using water washing and a hollow fiber filter. The bulk suspension was collected via filtration and lyophilized to obtain a free-flowing powder.

The process parameters and the characterization data for three representative batches (Batches 2-4) are shown in Table 2 in comparison to Vivitrol®:

TABLE 2

| Batch # | 2 | 3 | 4 | Vivitrol ® |
|---|---|---|---|---|
| Polymer | CHDM:TEG | CHDM:TEG:TEG-GL | CHDM:TEG:TEG-GL | PLGA |
| (Co-polymer | (93:7) | (88:10:2) | (88:10:2) | (75:25) |
| ratio) | | | | |
| MW (kDa) | 22 | 27 | 27 | Unknown |
| Solvent | DCM/BA | DCM/BA | DCM/BA | Unknown/BA |
| System | (3:1) | (3:1) | (3:1) | |
| Homogenizer | 3,250 | 3,250 | 4,000 | N/A |
| RPM | | | | |
| Drug Load | 49.6 | 49.7 | 49.1 | 33.7 |
| (% wt) | | | | |
| Residual | 0.1/3.5 | 0.1/3.4 | 0.1/2.8 | N.D./0.6 |
| Solvents (% wt.) | | | | |
| Particle Size | 13 | 14 | 13 | 50 |
| (D₁₀) | | | | |
| Particle Size | 29 | 30 | 26 | 81 |
| (D₅₀) | | | | |
| Particle Size | 52 | 52 | 45 | 129 |
| (D₉₀) | | | | |
| Microsphere | 22 | 28 | 28 | 74 |
| MW (kDa) | | | | |

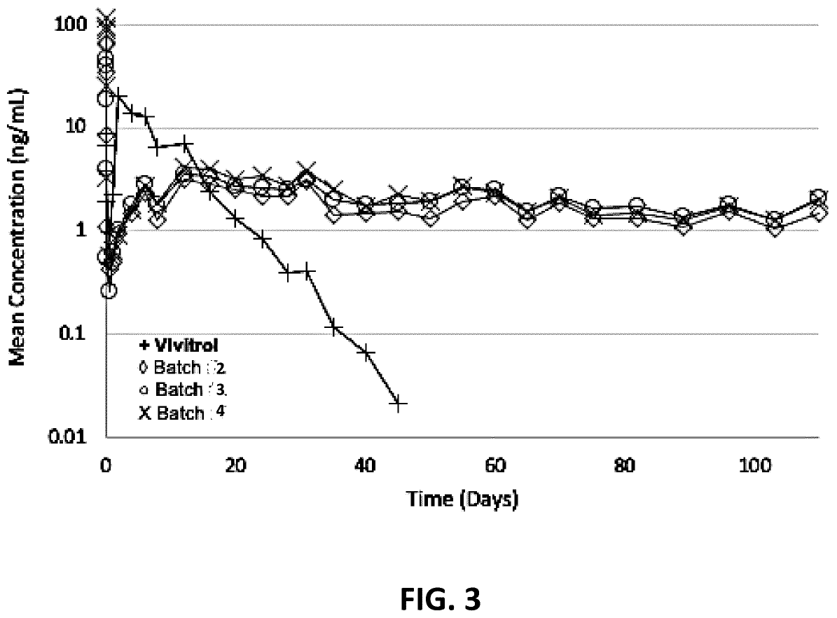

FIG. 3 is a graph showing naltrexone release over time in dogs from naltrexone-encapsulating POE polymer microspheres in direct comparison to Vivitrol®.

### Example 4—Preparation of Naltrexone-Encapsulated POE Polymer Microspheres—Batches 5-8

Following the general procedure described in Example 1, illustrated in FIG. 1, and detailed in Table 3, the DP was formed by dissolving 13.5 g of POE in 59.4 g of DCM and 19.8 g of BA (DCM/BA (3:1)), followed by addition of naltrexone (16.5 g) with mixing until completely dissolved. The DP was filtered and pumped at a flow rate of 25 mL/min into a Levitronix® BPS -i100 integrated pump system operating at 1,250 RPM (Batches 5 and 6) or 3,250 RPM (Batches 7 and 8). The CP comprising 0.35% PVA was also pumped into the homogenizer at a flow rate of 2 L/min (CP:DP=80:1).

The formed or forming microspheres exited the homogenizer and entered the SRV. Deionized water was added to the SRV. Solvent removal was achieved using water washing and a hollow fiber filter. The bulk suspension was collected via filtration and lyophilized to obtain a free-flowing powder.

The process parameters and the characterization data for four representative batches (Batches 5-8) are shown in Table 3.

TABLE 3

| Batch # | 5 | 6 | 7 | 8 |
|---|---|---|---|---|
| Polymer | CHDM:TEG:TEG-GL | CHDM:TEG:TEG-GL | CHDM:TEG:TEG-GL | CHDM:TEG:TEG-GL |
| (Co-polymer | (70:0:30) | (70:0:30) | (70:0:30) | (70:0:30) |
| ratio) | | | | |
| MW (kDa) | 20 | 20 | 20 | 20 |
| Solvent | DCM/BA | DCM/BA | DCM/BA | DCM/BA (3:1) |
| System | (3:1) | (3:1) | (3:1); | 1-hour EtOH 2% |
| | | | | solvent treatment* |
| Homogenizer | 1,750 | 1,750 | 3,250 | 3,250 |
| RPM | | | | |
| Drug Load | 50.6 | 40.7 | 49.0 | 48.4 |
| (% wt) | | | | |
| Particle Size | 23 | 18 | 11 | 11 |
| (D₁₀) | | | | |
| Particle Size | 43 | 38 | 25 | 20 |
| (D₅₀) | | | | |
| Particle Size | 75 | 69 | 52 | 35 |
| (D₉₀) | | | | |

*After the microsphere formation step, the microsphere suspension was dosed with 2% ethanol by volume and stirred for one hour. After stirring, the dosed microsphere suspension was concentrated and subjected to normal washing steps. This is intended to decrease the initial burst of the microspheres in vivo.

### Example 5—Preparation of Naltrexone-Encapsulated POE Polymer Microspheres—Batches 9-12

Following the general procedure described in Example 1, illustrated in FIG. 1, and detailed in Table 4, the DP was formed by dissolving POE in DCM and BA (DCM/BA (3:1)), followed by addition of naltrexone with mixing until completely dissolved. The DP was filtered and pumped at a flow rate of 25 mL/min into a Levitronix® BPS-i100 integrated pump system. The CP comprising 0.35% PVA was also pumped into the homogenizer at a flow rate of 2 L/min (CP:DP=80:1).

The formed or forming microspheres exited the homogenizer and entered the SRV. Deionized water was added to the SRV. Solvent removal was achieved using water washing and a hollow fiber filter. The bulk suspension was collected via filtration and lyophilized to obtain a free-flowing powder.

The process parameters and the characterization data for four representative batches (Batches 9-12) are shown in Table 4.

TABLE 4

| Batch # | 9 | 10 | 11 | 12 |
|---|---|---|---|---|
| Polymer | CHDM:TEG | CHDM:TEG | CHDM:TEG | CHDM:TEG |
| (Co-polymer | (80:20) | (80:20) | (80:20) | (80:20) |
| ratio) | | | | |
| MW (kDa) | 26.1 | 26.1 | 26.1 | 26.1 |
| Solvent | DCM/BA | DCM/BA | DCM/BA | DCM/BA |
| System | (3:1) | (3:1) | (3:1) | (3:1) |
| Homogenizer | 1,750 | 1,750 | 3,250 | 1,750 |
| RPM | | | | |
| Drug Load | 49.5 | 40.3 | 48.6 | 54.7 |
| (% wt) | | | | |
| Particle Size | 23 | 25 | 12 | 21 |
| (D₁₀) | | | | |
| Particle Size | 51 | 51 | 26 | 41 |
| (D₅₀) | | | | |
| Particle Size | 100 | 95 | 54 | 71 |
| (D₉₀) | | | | |
| Sample MW | 25.7 | 25.3 | 25.2 | 24.7 |
| (kDa) | | | | |
| Polymer MW | 26.1 | 26.1 | 26.1 | 24.2 |
| (kDa) | | | | |

### Example 6—Pharmacokinetics Study in Rats of Batch Nos. 3, 4, 9, 10, and 11 (the “PK study formulations”)

The pharmacokinetic profile of naltrexone following a subcutaneously injected dose of the PK study formulations in rats was studied. Five male rats per group (25 total rats) received a mg/kg dose (dose volume=1.5 mL/kg) of the stated Batch No. Blood was collected pre-dose, at 0.5, 1, 6, 12, 24, 48, and 96 hours, and at 7, 14, 21, 28, 35, 42, 49, 56, 63, 70, 77, 84, 91, 98, 105, 112, 119, 126, 133, and 140 days.

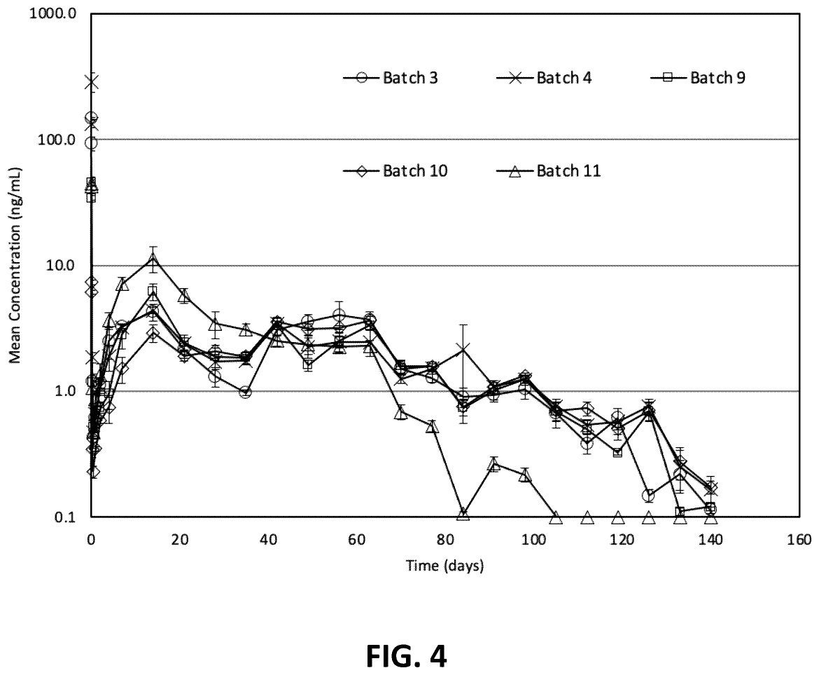

FIG. 4 is a graph showing in vivo release profiles of several naltrexone-encapsulating polymer microspheres. FIG. 4 shows a duration of release of about 60 days of a therapeutic level of naltrexone for Batch No. 11 and about 126 days for the other formulations.

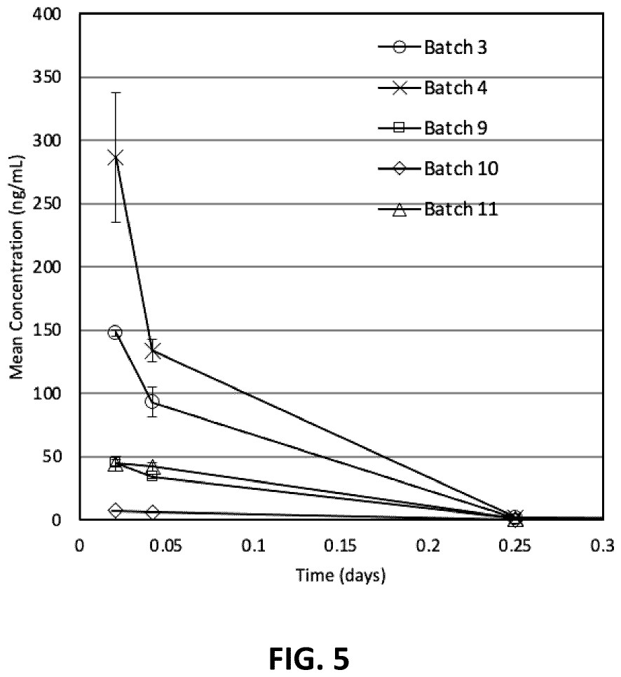

FIG. 5 is an exploded view of the graph shown in FIG. 4 and demonstrates the relative “burst” profiles of the naltrexone-encapsulating polymer microspheres. FIG. 5 shows that the 80:20:0 polymer (Batch Nos. 9-11) significantly decreased the burst compared to the 88:10:2 polymer (Batch Nos. 3 and 4).

### Example 7: Dosage

During the PK study described in Example 6, it was demonstrated that adjusting the dose based on a more linear release and a slight increase in drug loading helped to reduce injection volume. Table 5 compares dosage, total volume of diluent and microspheres, and microsphere concentration after re-concentration of Batch Nos. 9, 11, and 12 with Vivitrol. The change in polymer allowed for a more concentrated injection compared to Vivitrol.

TABLE 5

| | | | | | Total | |
|---|---|---|---|---|---|---|
| | | | | | Volume | MS |
| | | Drug | Particle | | of | Concentration |
| Duration | | Load | Size | Dose | Diluent + | after Recon. |
| (months) | Lot | (%) | (μm) | (mg) | MS (mL) | (mg/mL) |
| 1 | Vivitrol* | 33.7 | 81 | 380 | 4.0 | 282 |
| 2 | Batch | 48.6 | 26 | 760 | 3.9 | 400 |
| | No. 11 | | | | | |
| 3 | Batch | 54.7 | 41 | 760 | 3.5 | 400 |
| | No. 12 | | | | | |
| 4 | Batch | 49.5 | 51 | 760 | 3.8 | 400 |
| | No. 9 | | | | | |

*See https://www.vivitrol.com/content/pdfs/prescribinginformation.pdf; https://www.ncbi.nlm.nih.gov/pmc/articles/PMC7975983/

In use, the microspheres may be suspended in a diluent for administration (injection). The diluent may generally contain a thickening agent, a tonicity agent, and a surfactant. The thickening agent may include carboxymethyl cellulose-sodium (CMC-Na) or other suitable compounds. An appropriate viscosity grade and suitable concentration of CMC-Na may be selected so that the viscosity of the diluent is 3 cps or higher. Generally, a viscosity of about 10 cps is suitable; however, a higher viscosity diluent may be preferred for larger microspheres in order to minimize the settling of microspheres in the suspension.

Uniform microsphere suspension without particle settling will result in a consistent delivered dose during drug administration by injection. To have a tonicity of the diluent closer to the biological system, about 290 milliosmole (mOsm), solutes such as mannitol, sodium chloride, or any other acceptable salt may be used.

The aspects disclosed herein are not intended to be exhaustive or to be limiting. A skilled artisan would acknowledge that other aspects or modifications to instant aspects can be made without departing from the spirit or scope of the invention. The aspects of the present disclosure, as generally described herein and illustrated in the figures, can be arranged, substituted, combined, separated, and designed in a wide variety of different configurations, all of which are contemplated herein.

Unless otherwise specified, “a,” “an,” “the,” “one or more of,” and “at least one” are used interchangeably. The singular forms “a”, “an,” and “the” are inclusive of their plural forms. The recitations of numerical ranges by endpoints include all numbers subsumed within that range (e.g., 1 to 5 includes 1, 1.5, 2, 2.75, 3, 3.80, 4, 5, etc.). The terms “comprising” and “including” are intended to be equivalent and open-ended. The phrase “consisting essentially of” means that the composition or method may include additional ingredients and/or steps, but only if the additional ingredients and/or steps do not materially alter the basic and novel characteristics of the claimed composition or method. The phrase “selected from the group consisting of” is meant to include mixtures of the listed group.

When reference is made to the term “each,” it is not meant to mean “each and every, without exception.” For example, if reference is made to microsphere formulation comprising polymer microspheres, and “each polymer microsphere” is said to have a particular API content, if there are 10 polymer microspheres, and two or more of the polymer microspheres have the particular API content, then that subset of two or more polymer microspheres is intended to meet the limitation.

The term “about” in conjunction with a number is simply shorthand and is intended to include ±10% of the number. This is true whether “about” is modifying a stand-alone number or modifying a number at either or both ends of a range of numbers. In other words, “about 10” means from 9 to 11. Likewise, “about 10 to about 20” contemplates 9 to 22 and 11 to 18. In the absence of the term “about,” the exact number is intended. In other words, “10” means 10.

The term “therapeutic level” means the concentration of naltrexone, or a pharmaceutically acceptable form thereof, required to be present in a use environment (for example, in the blood) to provide effective treatment of a disease.

What is claimed is:

1. A microsphere formulation, comprising:
polymer microspheres, each polymer microsphere comprising:
naltrexone; and
a biodegradable polymer comprising a poly(ortho ester) (“POE”),
wherein each polymer microsphere comprises a drug load of naltrexone of greater than 40% by weight of the polymer microsphere, and
wherein the polymer microspheres have a particle size of between about 20 μm to about 60 μm (D₅₀).

2. The microsphere formulation of claim 1, wherein the naltrexone comprises naltrexone as a salt.

3. The microsphere formulation of claim 1, wherein the POE comprises a cyclohexanedimethanol:triethylene glycol (CHDM:TEG) co-polymer.

4. The microsphere formulation of claim 1, wherein the POE comprises a cyclohexanedimethanol:triethylene glycol (CHDM:TEG) co-polymer in a ratio of about 80: about 20.

5. The microsphere formulation of claim 4, wherein the polymer microspheres have a particle size of between about 25 μm (D₅₀) and about 50 μm (D₅₀)

6. The microsphere formulation of claim 1, wherein the POE comprises a cyclohexanedimethanol:triethylene glycol:triethylene glycol glycolide (CHDM:TEG:TEG-GL) tri-block polymer.

7. The microsphere formulation of claim 1, wherein the POE comprises a cyclohexanedimethanol:triethylene glycol:triethylene glycol glycolide (CHDM:TEG:TEG-GL) tri-block polymer in a ratio of about 88: about 10: about 2.

8. The microsphere formulation of claim 6, wherein the polymer microspheres have a particle size of about 20 μm to about 45 μm (D₅₀).

9. The microsphere formulation of claim 1, wherein each polymer microsphere comprises a drug load of about 45% to about 60% by weight of the polymer microsphere.

10. A pharmaceutical composition comprising the microsphere formulation of claim 1.

11. A microsphere formulation, comprising:
polymer microspheres, each polymer microsphere comprising:
naltrexone; and
a biodegradable polymer comprising a cyclohexanedimethanol:triethylene glycol (CHDM:TEG) co-polymer in a ratio of about 80: about 20,
wherein each polymer microsphere comprises a drug load of naltrexone of about 45% to about 60% by weight of the polymer microsphere, and
wherein the polymer microspheres have a particle size of between about 20 μm to about 55 μm (D₅₀).

12. A pharmaceutical composition comprising the microsphere formulation of claim 1.

13. A method for treating alcohol and/or opioid dependence, the method comprising administering by intramuscular or subcutaneous injection to a patient in need thereof the pharmaceutical composition of claim 12 no more frequently than about every 60 days.

14. A method for treating alcohol and/or opioid dependence, the method comprising administering by intramuscular or subcutaneous injection to a patient in need thereof the pharmaceutical composition of claim 12 no more frequently than about every 90 days.

15. A method for treating alcohol and/or opioid dependence, the method comprising administering by intramuscular or subcutaneous injection to a patient in need thereof the pharmaceutical composition of claim 12 no more frequently than about every 120 days.

16. A microsphere formulation, comprising:
polymer microspheres, each polymer microsphere comprising:
naltrexone; and
a biodegradable polymer comprising a cyclohexanedimethanol:triethylene glycol:triethylene glycol glycolide (CHDM:TEG:TEG-GL) tri-block polymer in a ratio of about 88: about 10: about 2,
wherein each polymer microsphere comprises a drug load of naltrexone of about 45% to about 60% by weight of the polymer microsphere, and
wherein the polymer microspheres have a particle size of between about 20 μm to about μm (D₅₀).

17. A pharmaceutical composition comprising the microsphere formulation of claim 16.

18. A method for treating alcohol and/or opioid dependence, the method comprising administering by intramuscular or subcutaneous injection to a patient in need thereof the pharmaceutical composition of claim 17 no more frequently than about every 120 days.

19. A method for treating an individual in need of naltrexone comprising the step of administering by intramuscular or subcutaneous injection a microsphere formulation comprising at least about 760 mg of naltrexone and a biocompatible polymer to the individual, wherein the serum AUC of naltrexone is about three times greater than that achieved by 50 mg/day oral administration, and wherein the biocompatible polymer is a poly(ortho ester) polymer (“POE”).

20. The method of claim 19, wherein the microsphere formulation comprises:
polymer microspheres, each polymer microsphere comprising:
naltrexone; and
the POE,
wherein each polymer microsphere comprises a drug load of naltrexone of greater than 40% by weight of the polymer microsphere, and
wherein the polymer microspheres have a particle size of between about 20 μm to about μm (D₅₀).
